(19) United States
(12) Patent Application Publication
(10) Pub. No.: US 2007/0106207 A1
(43) Pub. Date: May 10, 2007

(54) High-speed vaccination device

(76) Inventor: Edward L. Withey, Santa Barbara, CA (US)

Correspondence Address: MICHAEL G PETIT, 123 W PADRE ST., SUITE F, P O BOX 91929, SANTA BARBARA, CA, 93190, US

(21) Appl. No.: 11/412,010

(22) Filed: Apr. 25, 2006

Related U.S. Application Data
(60) Provisional application No. 60/733,190, filed on Nov. 4, 2005.
(60) Provisional application No. 60/734,012, filed on Nov. 7, 2005.
(60) Provisional application No. 60/740,507, filed on Nov. 30, 2005.

(51) Int. Cl. A61B 17/20; C12N 13/00; A61M 5/32; A61M 37/00

(52) U.S. Cl. 604/46; 604/272; 435/173.6

(57) ABSTRACT

A device for the rapid injection of a biological into the skin of a person. The device comprisies a hand-held applicator and a tape cassette adapted to be inserted into the applicator prior to use. The cassette houses a transportable tape having multiple doses of a biological disposed on a plurality of discrete clusters of microneedles affixed to the tape. In operation, a cassette is inserted into the applicator and a discrete cluster of microneedles advanced to a position adjacent a delivery opening in the applicator. A trigger advances the microneedle cluster to project outwardly through the delivery opening. The applicator is then pressed against the skin to force the cluster of microneedles into the skin. The applicator further includes means for applying an electric field between the microneedles and the skin to electrophoretically assist penetation of the biological into the skin. When the device is retracted from the skin and the trigger released, the next discrete cluster of biological-laden microneedles is advanced to underlie the delivery opening via a stepping tape transport means. The device may be used by relatively unskilled personnel to rapidly and painlessly vaccinate a large number of people.

This application claims the benefit of U.S. Provisional Application Ser. Nos. 60/733,190, filed Nov. 4, 2005, 60/734,012, filed Nov. 7, 2005 and 60/740,507, filed Nov. 30, 2005.

### BACKGROUND OF THE INVENTION

1. Field of the Invention

The present invention relates to a hand-held device operable for the high-speed intradermal delivery of a biological such as a vaccine to a patient. The device contains a plurality of doses of a vaccine releasably disposed on microneedle clusters affixed to a tape and can be used for vaccinating a plurality of people before refilling the device.

2. Prior Art

The human skin is comprised of several layers. The uppermost is the epidermis, covered by the stratum corneum, which serves as an effective mechanical barrier between tissues of the body and the outside environment. Cells populating the epidermis include keratinocytes, melanocytes, and especially important to vaccine delivery, Langerhans cells. Beneath the epidermal layer is the highly vascularized dermal layer, which nourishes the epidermis. The dermal layer includes blood vessels, nerves, lymph vessels, dendritic cells, hair follicles, collagen, and sweat glands. Underlying the dermal layer is is the fatty subcutaneous layer, with fat cells, blood vessels, and connective tissue.

Injections that are given subcutaneously or intramuscularly necessarily involve contact between the needle and nerves within the dermal layer and cause pain. To overcome this problem, microneedles and/or arrays of microneedles, such as porous silicon microneedles, are currently under development to create a delivery device that can deliver compounds to the interface between the stratum corneum (i.e., the barrier that prevents most topically-applied substances from being absorbed) and the dermal layer and thereby avoid impacting a nerve with a needle and causing pain.

In order to effect vaccination, a biological such as an antigen must pass from outside the skin to inside the skin wherein it is presented to cells of the immune system. Not only is the stratum comeum relatively impermeable, the antigen (biological) must traverse considerable skin tissue before the antigen is presented to cells of the immune system. Transdermal patches are effective for delivering small molecules to the interface between the epidermis and the dermis but are relatively ineffective for delivering larger molecules. Electrophoresis or iontophoresis can also be employed to improve the permeability of the stratum corneum. It would be an improvement in the art of vaccination if the transport of a biological into the interface between the epidermis and dermis did not require penetration of the stratum corneum by the antigenic substance.

U.S. Pat. No. 5,318,514 to Hofmann discloses an applicator for the electroporation of drugs and genes into cells. The applicator includes a plurality of needle electrodes which can be penetrated into the skin of a patient. Material to be electroporated into the skin is retained in a fluid reservoir which wets an open cell foam elastomer carrier for the fluid. Because the material to be electroporated is retained in a fluid, in both the reservoir and the open cell foam elastomer, careful control of the amount of the material at the electrode surfaces is difficult. It is difficult to control how much fluid flows down from the reservoir and the open cell foam elastomer to the surfaces of the needle electrodes, and, thereby, it is difficult to control how much of the treatment molecules is actually present on the surfaces of the needle electrodes as the electroporation process is being carried out on the patient. Moreover, the device lacks a cassette housing a plurality of doses of treatment molecules and further lacks a plurality of independent, discrete microneedle clusters disposed on a transportable tape. Accordingly, the device is inoperable for rapidly administering a single dose of treatment molecules to a large number of patients.

Microneedles have been known for many years. For example, U.S. Pat. No. 3,964,482 discloses the construction of microneedles. Commercialization of microneedle technology has been advanced by the recent development of inexpensive production methods as well as the identification of suitable production materials which produce strong microneedles that will overcome tissue penetration problems and that will not break easily. Radiation-sensitive polymers may be employed to fabricate microneedles. Polymeric microneedles can be coated, using electrochemical or sputtering techniques, with an electrically conductive material such as, titanium, gold and/or aluminum. These coated, electrically conducting microneedles can be used to enhance the permeability of the epidermis and dermis to facilitate drug delivery by employing electrophoresis: that is, passing an electric current between microneedles when the microneedles are at least partially embedded within the stratum comeum.

King et al., in pending U.S. Patent Application Pub. No. 2004/0203124, discloses the use of a (conductively-coated) microneedle assembly for the delivery of DNA vaccines to target cells, the DNA thereafter to be incorporated within the genome of the target cells. The apparatus employs a pulsed electric field having a defined waveform to increase penetration of the vaccine. Although the microneedle assembly is disposable, the device and method is limited to the administration of a single inoculation of DNA. Notwithstanding this limitation, the disclosure teaches the practicality of using a microneedle assembly for the intradermal delivery of a vaccine.

Although the recent developments in microneedle fabrication technology have improved the utility of microneedles for intradermal delivery of molecules such as vaccines, there remains a need for a multi-dose microneedle-based vaccination device, and a method for using the device for the mass vaccination of an at-risk population. Preferably, the device can be used by relatively unskilled healthcare workers for the rapid and painless administration of a vaccine to a large number of people. The present invention provides a multi-dose microneedle-based vaccinating device and a method for using the device to deliver a vaccine to cells adjacent the interface between the epidermal and dermal layers of the skin of an animal. While the device is discussed in the context of delivering a vaccine to people, it is understood that the device may be used for administering a vaccine to other animals as well.

### SUMMARY

The present invention is directed to a multi-dose vaccinating device and a method for using the device to administer a vaccine that substantially obviates one or more of the limitations of the related art. To achieve these and other advantages and in accordance with the purpose of the invention, as embodied and broadly described herein, the invention includes a hand-held applicator device that has a delivery opening and includes a multi-dose vaccine cassette removably housed within the applicator device. The cassette has a protective window that can be opened prior to use and houses a tape that has a plurality of discrete microneedle clusters disposed on an outer surface thereof, each cluster of microneedles having a single dose of vaccine coated thereon. The applicator device further includes a tape drive operable for positioning the tape such that a single cluster of microneedles underlies the protective (integrity) window in the cassette and the delivery opening in the applicator. When a trigger on the applicator is actuated, the delivery opening on the applicator is exposed and the cluster of microneedles affixed to the tape is advanced through the delivery opening on the applicator. When the applicator is pressed against a person's skin, the cluster of microneedles is driven into the epidermis of the person's skin. When the applicator is withdrawn from contact with the person's skin and the trigger released, a tape transport advances the tape such that a new cluster of microneedles is disposed to underlie the delivery opening in preparation for administering the vaccine to another person. An electrical pulse, or train of electrical pulses, may also be applied to the microneedles after they enter the skin. The force on the microneedle cluster pressed against the skin actuates an electrical pulse generator, causing an electrical pulse, or a pulse train to be applied to the microneedle cluster to assist penetration of the vaccine adhered to the microneedle cluster into the skin.

An essential feature of the present invention is the tape that supports and stores multiple doses of a vaccine. The tape has a length and a plurality of microneedle clusters affixed to a surface of the tape. The microneedle clusters are discretely disposed and equally spaced along the length of the tape. The microneedle clusters, which are preferably electrically conductive, have a therapeutic composition or a vaccine releasably attached to the microneedle clusters. The tape supporting the microneedle clusters and vaccine is wound on a delivery reel which is rotatably mounted within a cassette.

It is a further aspect of the invention to provide a method for making a tape having a plurality of microneedles clusters affixed to a surface thereof and a method for releasably coating the microneedle clusters with a vaccine.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a side elevational view of a vccine applicator device in accordance with a preferred embodiment of the present invention.

FIG. 2 is a top plan view of a vaccine cassette adapted to be removably. housed within the cassette compartment of FIG. 3.

FIG. 3 is a top plan view of a cassette compartment within the applicator of FIG. 1, the cassette shown in phantom within the compartment. The cassette compartment is accessed through a door in the outer wall of the applicator. When the cassette is correctly positioned within the cassette compartment, the tape reels are engaged by tape transport mechanisms in the applicator.

FIG. 4 is a top plan view of a cassette compartment in accordance with a preferred embodiment of the invention, showing the position of the components contained therein prior to inoculation.

FIG. 5 is a top plan view of a cassette compartment in accordance with FIG. 4, showing the position of the components contained therein during inoculation of a patient with a vaccine.

FIG. 6 is a side view of a mechanical embodiment of an integrity door opening assembly.

FIG. 7 is a top plan view of a section of tape having a plurality of microneedle clusters affixed to an upper surface thereof.

FIG. 8 is an enlarged side view of a microneedle cluster.

FIG. 9 is a side view of a roller illustrating a recessed portion in the roller to prevent contact of the microneedle cluster on the tape with the surface of the roller.

FIG. 10 is a plan view of an exemplary embodiment of a tape strain relief mechanism which can be employed to prevent rupture of the tape when the applicator is actuated and the tape backing plate (and the portion of the tape supporting a microneedle cluster) is advanced through the delivery opening in the applicator.

FIG. 11 is a top view of a section of a protective tape bearing a plurality of discrete, equally-spaced apertures wherein the apertures are positioned to overlie microneedle clusters that project upwardly from the vaccination tape when the protective tape is brought into registered juxtaposition with the vaccination tape of FIG. 12.

FIG. 12 is a top view of the tape having microneedle clusters affixed to discrete segments of conductive film that is protected by the tape set forth in FIG. 11.

FIG. 13 is a schematic view of a first embodiment of an apparatus for making a tape having a plurality of discrete, electrically conductive, vaccine-coated microneedle clusters affixed thereto.

FIG. 14 is a schematic view of a second embodiment of an apparatus for making a tape having a plurality of discrete, electrically conductive, vaccine-coated microneedle clusters affixed thereto.

FIG. 15 illustrates, in top 15a and side 15b view, the general shape of a pocket in a vaccination tape wherein the pocket is recessed to accommodate a microneedle cluster (not shown) therein.

### DESCRIPTION OF THE PREFERRED EMBODIMENTS

The invention described herein provides a device operable for the rapid and painless delivery of a vaccine to a large number of people by relatively unskilled personnel. The invention also provides a method for storing multiple doses of a vaccine on a plurality of discrete microneedle clusters affixed to a tape housed within a cassette. The vaccine is stored on the outer surface of the microneedles comprising the linearly-spaced microneedle arrays (clusters), and rolled on a reel of plastic tape. The reels are housed in cassettes which fit into a hand-held delivery device (applicator) which operates automatically to drive the microneedles comprising a particular cluster into the epidermis when the operator presses the applicator against the skin of a patient and actuates a trigger. The applicator, alternatively referred to herein as “delivery device”, includes a tape transport means and either an integral power supply, or means for connecting the applicator to an external power supply. The applicator further includes sequencing logic which controls the tape position for vaccine delivery and automatically prepares the applicator for the next injection cycle.

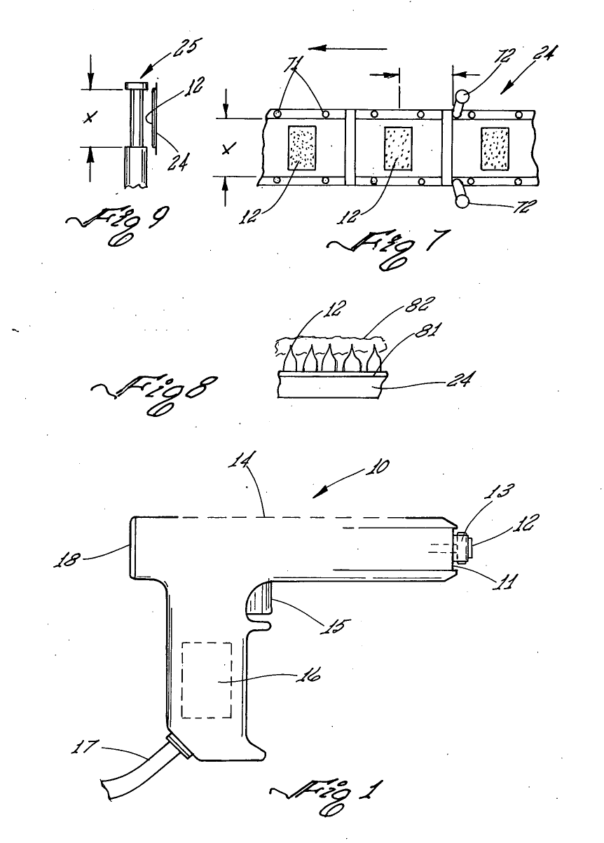

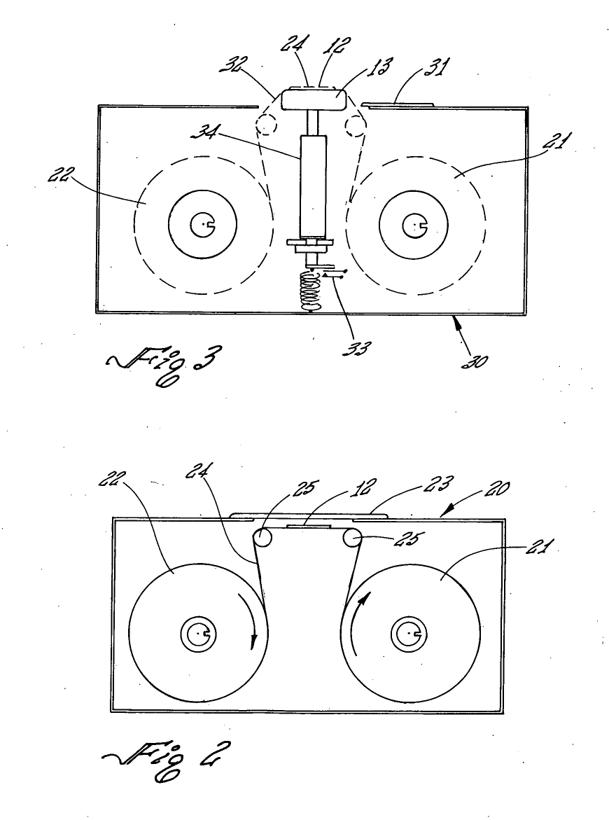

Turning now to FIG. 1, a representative embodiment of an applicator (delivery device) in accordance with the present invention is shown in side elevational view at numeral 10. While the applicator 10 is intended to be hand-held, it will be obvious to the artisan that the size and shape of the applicator 10 may vary in order to accommodate the tape cassette and the particular choice of tape transport mechanism, power supply and mechanical actuators selected for the operation of the applicator 10. The applicator 10 has an openable delivery port 11 through which an array (cluster) of microneedles 12, supported by a microneedle backing plate 13, project. The applicator 10 includes a cassette access door 14 which provides means for mounting a vaccine cassette 20 (FIG. 2) in a cassette receptacle 30 (FIG. 3) or, more preferably, the cassette compartment indicated at 40 in FIG. 4, recessed within the applicator 10. The applicator 10 further includes a trigger assembly 15 and either an integral power supply 16 (shown in phantom) or a cable 17 operable for electrical connection of the applicator 10 to an external power supply (not shown). The applicator 10 preferably further includes a viewable readout 18 indicating the number of doses of vaccine remaining in the applicator.

A vaccine cassette 20 for use with the applicator 10 is shown in top plan view in FIG. 2. The cassette 20 has a supply reel 21 and a take-up reel 22 rotatable mounted therein in a manner well known in the art. A slidable front cover or openable protective window 23 provides protection of the tape 24 and the microneedle cluster(s) 12 affixed to the tape from the external environment prior to use. The tape 24 is wound on the supply and take-up reels in a manner similar to a VHS video cassette. The tape 24 has a plurality of discrete microneedle clusters 12 affixed to an outer surface thereof (only a single microneedle cluster 12 is shown in FIG. 2 for clarity). The tape 24 is drawn off of supply reel 21, travels over tape guides 25, and, after use, is wound onto take-up reel 22.

With reference to FIG. 3, a simple embodiment of the cassette compartment 30 within the applicator 10 is shown in top view with portions of the cassette 20 (shown in phantom) positioned within the compartment 30. The compartment 30 has a retractable integrity cover 31 that is shown in an open position to expose delivery opening 32. A portion of the tape 24 bearing a cluster of microneedles 12 is centrally disposed within the delivery opening 32 forward of the backing plate 13. A solenoid 34, illustrated in an activated position, thrusts backing plate 13 forwardly, advancing the microneedle cluster 12 through the delivery opening 32 in response to an actuating signal from switch 15. As the cluster of microneedles 12, laden with vaccine, advances through the opening 32, the cluster of microneedles penetrate the skin of a patient (not shown), depositing the vaccine therein. While the embodiment of the cassette compartment indicated at numeral 30 teaches the general operation of the cassette compartment components, the embodiment 30 is not suitable for use in the applicator due to the potential for damage to the tape during operation of the applicator.

A more preferred embodiment of a cassette compartment is illustrated in top view at numeral 40 in a nonactivated position (FIG. 4) and an activated position (FIG. 5). With reference first to FIG. 4, a cassette compartment disposed within an applicator 10 in accordance with a preferred embodiment of the present invention is indicated at numeral 40. Prior to activation (i.e., prior to the administration of a dose of vaccine) the vaccination assembly 41, mounted within compartment 40 of the applicator 10 (not shown in FIG. 4) between supply reel 21 and take-up reel 22, is disposed behind the openable integrity door 31. The vaccination assembly 41 has a wheel 42 rotatably mounted on the foremost end of a shaft 43 which is slidably mounted within tube 46. The tube 46 is constrained to linear motion by guides AA. The tube 46 bears a rack gear which is engaged by pinion 44 driven by stepping motor 45. A pin 48 affixed to shaft 43 rides in a vertical slot 47 in the wall of tube 46 to constrain the travel of the shaft. Tube 46 also includes contact arms 49 and 49′ which can engage contacts 50 and 50′ which serve as limit switches to signal the controller when the wheel 42 is extended through the delivery opening 32 (FIG. 5) by closure of switch 50, or fully retracted within the compartment (as shown in FIG. 4) by closure of switch 50′. When the wheel 42 is extended, as shown in FIG. 5, contact 50 closes to contact 49′. When wheel 42 is retracted, contact 49 closes with contact 50′. A spring 49b bears between the lower end of tube 46 and the lower end of shaft 43, urging the shaft upward. The lower end of tube 46 also bears an insulated contact 49a that is coaxial with, but insulated from, spring 49b. When the trigger 15 on the applicator 10 is pulled, and vaccination wheel 42 is pressed against the patient's skin, spring 49b compresses, allowing contact 49a to close against the lower end of shaft 43 which signals the controller to initiate the application of an electrical pulse or pulse train to the microneedle cluster to effect the delivery of vaccine to the patient. When the trigger 15 is released, the stepping motor 45 is then operated to retract wheel 42 and tape transport means advances the tape to position the next microneedle cluster adjacent the delivery opening 32.

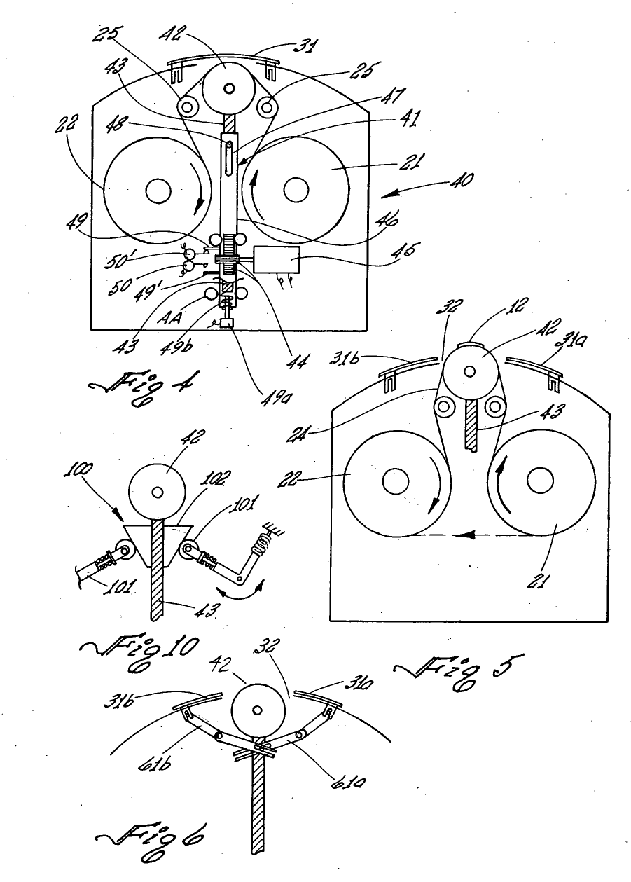

An example of a mechanical door-opening means operable for opening the integrity door 31 to expose the delivery opening 32 as the shaft 43 and wheel 42 advances is illustrated in FIG. 6. The integrity (delivery) door 31 is in two parts: 31a and 31b, each part being slidably mounted on the cassette compartment 40 to expose (FIG. 5) or occlude (FIG. 4) the delivery opening 32. Parts 31a and 31b of the integrity (delivery) door 31 include projections which ride in slots (not visible in FIG. 6) in the applicator 10 housing. Arms 61a and 61b are pivotally mounted to the shaft 43 at a medial end thereof and to parts 31a and 31b at a lateral end thereof. As the shaft advances toward the delivery opening, the parts 31a and 31b slide laterally to expose the delivery opening 32 enabling the wheel 42 to extend through the delivery opening. As the shaft retracts, the door parts 31a and 31b are urged together to occlude the delivery opening 32. Of course, it will be obvious to the artisan that springs may be used to facilitate opening or closing of the integrity (delivery) door 31. A solenoid or similar electromechanical device may be used as well for opening and closing parts 31a and 3lb.

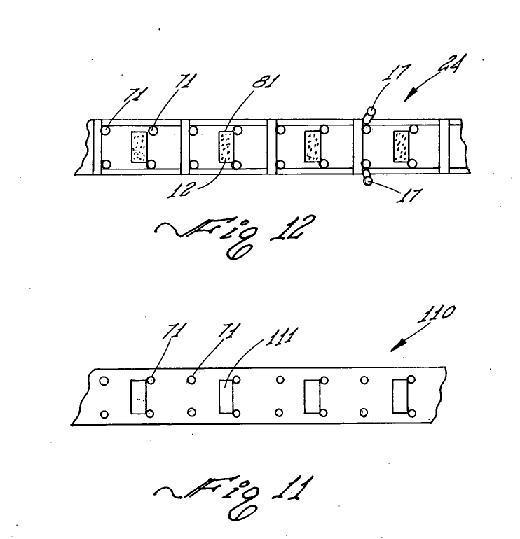

The tape that is employed to support, store and transport a plurality of microneedle clusters having vaccine on a surface thereof is a critical part of the present invention. Turning now to FIG. 7, a section of tape 24 bearing a plurality of evenly-spaced microneedle clusters 12 disposed on an upper surface thereof is shown in top view. An enlarged side view of a microneedle cluster 12 supported by a tape 24 is illustrated in FIG. 8. Each of the microneedle clusters 12 are deposited on a discrete layer or film of a conductive material 81 applied to the upper surface of the tape 24. The individual microneedles comprising each microneedle cluster 12 are either electrically conductive or coated with an electrically conductive layer. A vaccine 82 (FIG. 8) is applied to the microneedle cluster to overlie the conductive coating and microneedle cluster thereon. The lateral edges of the tape 24 include a plurality of evenly spaced perforations 71 adapted to engage a rotating motor-driven sprocket to facilitate tape transport in a manner well known in the art. Wipers 72 maintain contact with the metalized portions of the tape and provide a signal to indicate tape position and to conduct electroporation pulse(s) to the microneedle clusters.

It should be noted that since the tape 24 has a plurality of sharp microneedle clusters 12 projecting from a surface of the tape, care must be taken not to dull or break the microneedle clusters 12 during tape transport and storage. Accordingly, it is desirable to employ guide rollers 25 having a recessed portion, indicated at X in FIG. 9, to underlie the microneedle clusters 12 as the tape is transported over the guide roller. It should also be noted that when the applicator 10 is actuated, the microneedle cluster overlying the backing plate is advanced through the delivery opening in the applicator which results in the application of tension to the tape. If the tape guide rollers 25 are supported by a strain relief mechanism such as illustrated in FIG. 10, as the shaft 43 is advanced, guide roller support(s) 101 travel inwardly as they ride along the conical surface of a cam 102 attached to the shaft. The inward travel of the guide roller support(s) 101 during advancement of the shaft 43 is sufficient to maintain constant tension on the tape (not shown in FIG. 10) during advancement of shaft 43 and roller or wheel 42 (FIGS. 4 and 5).

Process for Making Tape Having a Plurality of Microneedle Clusters Disposed on a Surface Thereof.

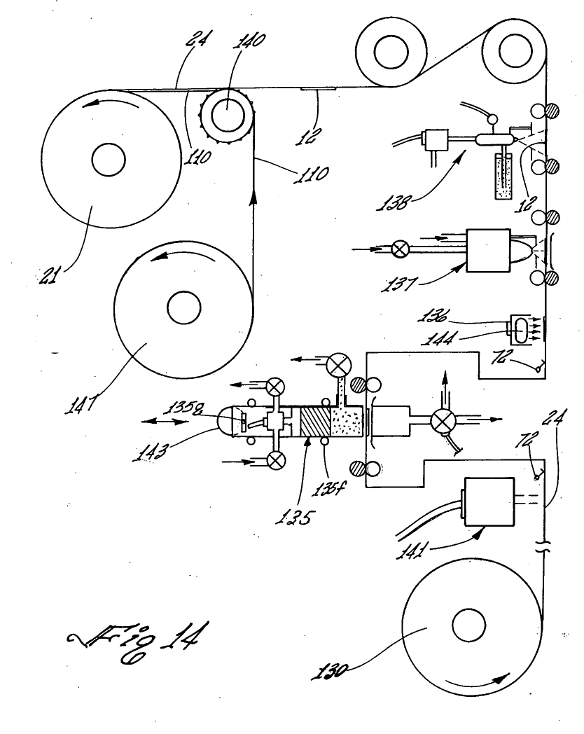

Three approaches for the production fabrication of the storage reels 21 containing vaccine are presented. With reference now to FIG. 13, which is a schematic view of a first process for fabricating a tape in accordance with the present invention, tape supply reel 130 feeds tape 24 between embossing wheels 131 and 132 which contain mating male an female dies disposed along the outer circumference thereof. As the tape 24 passes therebetween, a pocket, or intaglio-type depression 133 is created in the tape 24. If a thermoset tape is used, wheels 131 and 132 can be supplied with internal heaters and thermostats powered by slip rings (not shown) to heat the tape. The purpose of forming the pockets 133 in the tape, as shown in detail in FIG. 15, is to allow the tape, with the formed microneedle clusters recessed within the pocket 133, to be wound on reels without turn-to-turn damage to the needles. This is effected by making the depth of the pocket, H in FIG. 15, greater than the height of the microneedles, which are typically ˜0.1 to 0.8 mm. in length. The dimensions of the microneedle clusters 12 in FIG. 8 are greatly exaggerated in order to better illustrate the construction of the microneedle cluster 12. Also, in FIGS. 13 and 14, the tape spacing between the processing steps (stations) is relaxed for clarity.

After passing between the pocket-forming dies 131 and 132, the tape 24 passes to spray station 134, which is a conductive film spray station. Stepping motors (not shown) advance the tape 24 to a position wherein the pocket 133 is disposed before the spray head 134a. The spray station 134, which deposits a film of electrically conductive material such as a hot or cold metal powder in the pocket 133 through a spray mask 134e, includes a powdered metal inlet 134b, a gas inlet 134c that forces a gas through a (normally closed) solenoid-actuated valve X. A power cable 134d which is connected to a programmable process sequencer (not shown), controls the spraying of metallic film into the pocket 133. An insulated, shaped, electrically conductive backing plate 134f is disposed behind the tape 24 and connected to a voltage source (not shown) through the programmable process sequencer. The backing plate 134f can be pulsed to a high positive voltage in synchrony with a pulse to the gas solenoid X to effect a controlled spray time of the metallic film into the pocket 133 in the tape.

After the conductive film is deposited into the pocket 133 to form a metallized pocket, a tape transport means such as a stepping motor advances the tape 24 until the metallized pocket 133 is disposed in front of a needle forming station 135. The registerable positioning of the pocket 133 at any particular station during the process is facilitated by including position-sensing wiper(s) 72 along the feed path of the tape to stop the tape transport mechanism when the pocket 133 in the tape 24 is correctly positioned. As the tape is advanced to the needle forming station 135, wipers 72 detect a metallized segment on tape 24 and stop the further advance of the tape with the pocket 133 facing extruder head 135c. Needle forming station 135 is an extruder comprising a piston 135p slidably disposed within a cylinder 135a, an extrusion head 135c, a 3-way solenoid valve 135d, and extrudable material charging means such as a pump operable for charging the portion of the cylinder 135a between the piston 135p and the extrusion head 135c with a photopolymeric paste through check valve 135e. The entire needle forming station 135 is slidably mounted and constrained to move along an axis (indicated by the double-headed arrow) by guides 135f.

A cam pin 135g mounted off-center on a motor shaft at 143 engages the horizontal slot (not numbered) in the body of the extruder adjacent motor shaft 143. When the stepping motor (not shown) is pulsed to rotate the cam motor shaft 143 180 degrees, the extrusion head 135c is brought proximal to the metalized pocket. The extrusion head 135c contains a plurality of holes; the diameter of the holes being equal to the desired diameter of the microneedles, and when a pressure pulse of gas or fluid from gas source 135h is passed by solenoid valve 135d into the portion of the cylinder 135a rearward of the piston 135p, the piston 135p is urged toward the extruder head 135c and the polymer is extruded onto the metalized pocket forming a cluster of cylindrical needles. At the end of the extrusion cycle, the motor shaft is again pulsed to turn the cam 180 degrees, moving the extruder head 135c away from the pocket and drawing the polymer away from the base of the cylindrical needle to form an essentially “sharpend” tip on the (cylindrical) microneedles as shown in FIG. 8 (the microneedles being exaggerated to illustrate the concept).

With continued reference to FIG. 13, when the pocket 133 in the tape is in position for extrusion of the microneedle cluster, a 2-way solenoid valve 135k connects vacuum line 135n to vacuum chamber 135q. The portion of the tape containing pocket 133 is drawn against an apertured backing plate 135m and held firmly in position for extrusion of the microneedle clusters. As stated above, cam shaft 143 rotates to position the extruder head adjacent the pocket 133. When the extruder head 135c is fully advanced, 3-way solenoid valve 135d then opens to pressure source 135h allowing pressure to bear against piston 135p, forcing the photopolymeric paste (stippled) in cylinder 135a through the cluster of openings in the extruder head 135c to form the needles. After the cluster of substantially cylindrical needles are extruded, cam shaft 143 again rotates, withdrawing the assembly 135 away from the pocket in a controlled fashion to draw out the (viscous) extruded photopolymer to form tips on the needles. After the sharp tips are formed on the needles, valve 135d opens to the atmosphere to relieve the extrusion pressure and, simultaneously, valve 135k opens to vent the vacuum chamber 135q to the atmosphere thereby releasing the tape from the apertured backing plate 135m so that the pocket containing the freshly extruded clusters can be transported to the needle hardening station 136. High voltage pulses can be applied in synchrony with the extrusion of the needles to the backing plate 135m and/or the metallized pocket 133 to facilitate needle formation. Pulses necessary to effect tape transport, reciprocal movement of the extruder assembly comprising needle forming station 135, valve control and high-voltage pulsing are preprogrammed in the sequencing microprocessor and control power supply. All tubing and connections to the extruder 135 must be sufficiently flexible to permit reciprocal motion of the extruder assembly comprising needle forming station 135.

The tape is then moved such that the pocket bearing the cluster of microneedles is adjacent a needle hardening station 136, which may be a UV source, or sources, which both sets the polymer and sterilizes the polymeric microneedles. The vaccination tape 24 is then moved to a second spray station 137, which is similar or identical to spray station 134, where the polymeric microneedles are metalized by application of an electrically conductive coating thereto by the same process as previously described for depositing a metallic film in the pocket 133.

Following metallization of the microneedle clusters at the second spray station 137, the tape is advanced such that the electrically conductive microneedle cluster that is affixed to the metallized film in the pocket is adjacent the vaccine spray coating station 138 where the vaccine is spray-coated onto the metalized cluster of microneedles. Vaccine spray head 138a connects to a gas source 138b through solenoid valve 138c, and to container 138d holding vaccine 138e. A spray baffle 138f is connected to spray head 138a and to a high voltage terminal 138g. The vaccine spray head 138a is electrically isolated and can be pulsed to a high voltage in synchrony with pulsing the valve 138c open, effecting a pulsed spray onto the microneedle cluster which is grounded by the guides 139a and 139b. This vaccine coating method embraces a wide range of options that include the choice of gas, gas pressure, pulse “on” time, and magnitude and width of the high voltage pulse sufficient to insure that the microneedle cluster can be coated with a minimum of vaccine. This can be of critical importance with a vaccine that is both expensive and in short supply. The finished tape is now guided to the take-up reel 21, where it can be stored until needed for the vaccination unit previously described. The process for making a tape in accordance with FIG. 13 assumes fabrication from a standard plastic tape, purchased with the desired width and thickness, and with properties appropriate to the pocket forming step—which may include thermoplastic characteristics.

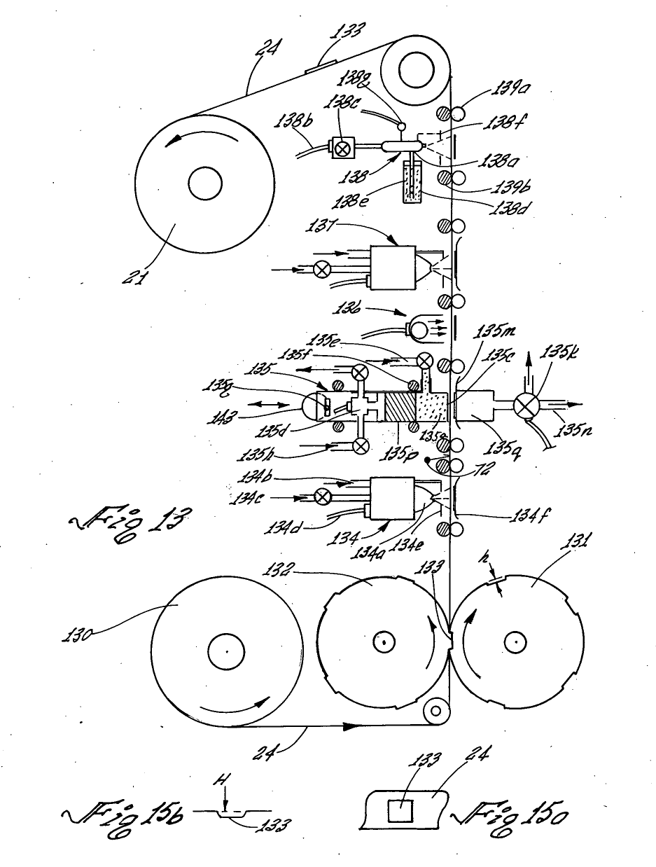

A second process, also designed to protect the needle array from turn-to-turn mechanical damage during winding without requiring the microneedle cluster(s) to be contained within a pocket, is shown in FIG. 14. In this second process, a protective tape 110 (a portion of the protective tape is shown in top view in FIG. 11) and a standard tape 24 of the desired width, thickness and having a metallic film deposited thereon is purchased from the supplier with a specification for the shape and spacing of sprocket holes, both to control movement and to insure registration of the metalized vaccination tape 24 with the protective tape 110, shown in top view in FIG. 11. The protective tape 110 has sprocket holes 71 and cut-outs or apertures 111 dimensioned and spaced to matingly overlie the microneedle clusterl2 on vaccination tape 24 (FIGS. 7 and 12). Sprocket drive mechanisms (not shown) operable for transport and synchronization of the vaccination tape as it moves from station to station during the fabrication process, is a mature technology, refined especially for motion picture film.

With continued reference to FIG. 14, supply reel 130 supplies the metalized and sprocket punched vaccination tape 24 which is controllably moved through the fabrication process by sprocket wheel 140, driven by a stepping motor (not shown). The vaccination tape 24 is advanced from the supply reel 130 to a stripping station 141 which removes bands of metal from the metalized tape to create isolated, electrically conductive metal frames which can then be detected by wipers 72 to control the stepping motor drive on sprocket 140. The stripping station 141 is preferably a pulsed scanning laser, such as a C02 laser, which can be controlled to vaporize targeted portions of the metalic film on the vaccination tape 24.

The vaccination tape 24 is then moved to a microneedle forming station 135 which comprises an extruder as previously described which is slidably mounted and constrained by guides 135f to move back an forth in the direction of the double-headed arrow in response to a 180 degree rotation of a carn arrangement 135g and 143 as previously described. Alternatively, the extruder 135 may be replaced with an inkjet printer employing ink jet printing technology, and bearing a matrix of printing orifices having a diameter equal to the diameter of the desired microneedles. The inkjet printer can be connected by cable to a print driver and a computer card in the control and sequencing unit. Extruder 135 is charged with a photo-setting polymer, which may be set by UV, visible or IR radiation. As previously described, the cam arrangement 135g brings the extruder head proximal to the vaccination tape 24, extrudes a cluster of cylinders, and, at the end of the extrusion cycle, the cam shaft 143 and cam pin 135g mounted thereon rotates 180 degrees and moves the extruder head assembly away from the vaccination tape 24, drawing the (unset) cylinder tips to a fine point as shown in FIG. 8. A simultaneously pulsed electric field can be applied to aid polymer transfer. The vaccination tape is then moved to setting station 136 which comprises a radiation source which may emit either UV, visible or IR as required to set the polymer.

After the microneedles are formed and cured, the vaccination tape 24 is moved to the spray metalizing station 137 as previously described, in order to metalize the needle array. The vaccination tape 24 is finally advanced such that the microneedle cluster is adjacent vaccine applying station 138. The vaccine is sprayed onto the metalized microneedle cluster as previously described. Following application of the vaccine to the microneedle cluster and drying, the vaccination tape 24 is guided to the sprocket wheel 140 where the protective tape 110 is unwound from protective tape supply reel 147 and brought into registration contact with the vaccination tape 24 and wound onto the storage reel 21. The sprockets engage the sprocket holes 71 (FIGS. 11 and 12) on both the vaccination tape 24 and the protective tape 110 to insure perfect registration of the cutouts 111 with the microneedle clusters 12 during winding of the laminate onto the storage reel 21 to protect microneedles from damage during winding and storage.

All of the electrical operations required for tape transport, as well as their synchronism with controlling the tape movement, are well known to those skilled in the art. The operation of the tape transport mechanism generally involves the generation of precise pulse trains to operate stepping motors, triggering high voltage pulses of controlled width and amplitude, pulsing the solenoid valves in the proper sequence, pulsing radiation sources and all other control functions. The control signals can be provided by a single programmable microprocessor. Power for the control module can be supplied by an internal battery, by an external direct current 12 volt supply or by a 120-230 volt, 50-60 Hz source of power.

Two approaches that are operable for the (essentially continuous) production and storage of a vaccination tape supporting a plurality of discrete microneedle clusters, each microneedle cluster bearing a dose of vaccine, have been disclosed. A third process for forming the vaccination tape consists of fabricating suitable large-scale microneedle arrays disposed on a substrate by means of a batch process such as extrusion, vacuum sputtering or deposition, photoetching or the like. The individual microneedle clusters and supporting substrate can be cut from the array and attached to the tape by adhesive means. Suitable spacing between microneedle clusters on the tape can be maintained by positioning means that are well known in the art.

In each of the processes, the vaccination tape 24 is rolled onto a reel and stored in a cassette for use. An instrument (applicator 10) for accepting these prepared cassettes and delivering the vaccine to patients rapidly and painlessly by unskilled personnel is also disclosed herein. The systems and technologies disclosed herein are believed to meet or exceed the future requirements of mass vaccination. It is recognized that vaccines have a shelf-life that may vary from vaccine to vaccine. In the event that a vaccine has a particularly short shelf-life, immediatley prior to use, a cassette having a vaccination tape therewithin, but lacking a vaccine coating on the microneedle clusters, can be inserted into a vaccine coating apparatus (not shown) such as the spray station 131, and the vaccine coating applied to the microneedle clusters. The vaccination tape 24, thus coated, is then rewound to prepare the cassette for insertion into an applicator. Further, it is contemplated that a plurality of microtubes or nanotubes may be deposited on the surface of a substrate to form a “hairy” surface, a portion of which is suitable for providing a microneedle cluster as described hereinabove. While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What I claim is:

1. A tape having a length and a plurality of microneedle clusters affixed to a surface of said tape, said microneedle clusters being discretely disposed and equally spaced along said length of said tape.

2. The tape of claim 1 further comprising a therapeutic composition releasably attached to said microneedle clusters.

3. The tape of claim 2 wherein said therapeutic composition is a vaccine.

4. The tape of claim 3 wherein said tape is wound on a delivery reel.

5. The tape of claim 4 wherein said delivery reel is rotatably mounted within a cassette.

6. The tape of claim 1 wherein said microneedle clusters have an electrically conductive layer on an outer surface thereof.

7. The tape of claim 6 further comprising a therapeutic composition releasably attached to said microneedle clusters.

8. The tape of claim 7 wherein said therapeutic composition is a vaccine.

9. The tape of claim 8 wherein said tape is wound on a delivery reel.

10. The tape of claim 9 wherein said delivery reel is rotatably mounted within a cassette.

11. The tape of claim 1 wherein said microneedle clusters are formed from an electrically conductive material.

12. The tape of claim 11 further comprising a therapeutic composition releasably attached to said microneedle clusters.

13. The tape of claim 12 wherein said therapeutic composition is a vaccine.

14. The tape of claim 13 wherein said tape is wound on a delivery reel.

15. The tape of claim 14 wherein said delivery reel is rotatably mounted within a cassette.

16. A hand-held vaccine applicator device operable for administering a dose of a vaccine to an animal, said applicator comprising:
(a) a cassette compartment operable for releasably receiving a tape cassette having a plurality of doses of a vaccine releasably adhered to a plurality of discrete microneedle clusters disposed on a tape housed within said cassette;
(b) an openable delivery opening in said applicator;
(c) tape transport means operable for transporting said tape to position a portion of said tape having a single microneedle cluster with a dose of vaccine thereon adjacent said delivery opening;
(d) first actuator means operable for: (i) opening said openable delivery door, and (ii) advancing said portion of said tape having a dose of vaccine thereon through said delivery opening to press against the animal's skin such that microneedles comprising said microneedle clusters penetrate the epidermis of the animal's skin and release at least a portion of said vaccine beneath the epidermis.
(e) means for advancing said tape after the release of a dose of vaccine such that an unused microneedle cluster is positioned adjacent said delivery opening

17. A vaccine applicator in accordance with claim 16 further comprising a second actuator operable for applying a voltage to said microneedle cluster after said microneedle cluster penetrates the epidermis of the animal's skin.

18. A method for making a tape having a length and a plurality of discrete microneedle clusters disposed on an upper surface of the tape along said length of said tape comprising the steps of:
(a) presenting a tape having a length and an upper surface;
(b) presenting a substrate having an upper and lower surface and an array of microneedles disposed on said upper surface thereof;
(c) removing a portion of said substrate containing said array of microneedles, said removed portion defining a single cluster of microneedles; then
(d) adhering said lower surface of said removed portion to said upper surface of said tape.

19. The method of claim 18 further including the step of adhering a vaccine to said microneedle clusters adhered to said tape.

20. The method of claim 18 wherein said microneedles are electrically conductive.

21. The method of claim 20 further including the step of adhering a vaccine to said microneedle clusters adhered to said tape.
